(19) United States
(12) Patent Application Publication
(10) Pub. No.: US 2024/0183839 A1
(43) Pub. Date: Jun. 6, 2024

(54) PROBE AND METHOD FOR COLLECTING DATA ON CURING CONCRETE

(71) Applicant: Invisense AB, Linköping (SE)

(72) Inventors: Björn Garplind, Linköping (SE); Jan Strandberg, Linköping (SE); Jörgen Blomqvist, Vreta Kloster (SE)

(21) Appl. No.: 18/287,790

(22) PCT Filed: Apr. 22, 2022
(86) PCT No.: PCT/EP2022/060674

§ 371 Date: Oct. 20, 2023

(30) Foreign Application Priority Data
Apr. 22, 2021 (SE) 2150500-3

(51) Int. Cl. G01N 33/38

(52) U.S. Cl. CPC G01N 33/383

(57) ABSTRACT

A sensor device (1, 1′) for collecting data on a material in which the sensor device is inserted on concrete during a curing process of said concrete, comprises an elongate sensor body (10), extending along a longitudinal direction (L), and having a head end (102) and a tip end (101), at opposite axial ends of the sensor body (10); at least two first sensor elements (131a, 131b, 131c), which are spaced apart along the longitudinal direction (L), both of which being configured for sensing at least one first parameter relating to the concrete material at a respective position along the longitudinal direction (L), and a locking surface (11b) which faces towards the head end, an exterior thread extending helically along at least a portion (102) of the sensor body (10).

### TECHNICAL FIELD

The present disclosure relates to a probe and a method for collecting data on concrete while curing.

### BACKGROUND

In various building application involving casting of concrete structures, such as concrete constructions, such as slabs, joists, pillars, beams, walls or other structures, there is a desire to be able to follow the process of the concrete setting and hardening, in order to ensure correct curing of the concrete and in order to determine when the concrete has cured sufficiently to allow further construction.

It is known from e.g. CN111505252A to monitor temperature and humidity of the concrete while it is setting or curing.

However, there is a need for a system which enables simple and user-friendly monitoring of a curing concrete structure, while providing more detailed data on the curing process of the concrete.

Hence, there is a need for improvements in the data collection on concrete during its setting and hardening process.

### SUMMARY

It is an object of the present disclosure to improve the data collection on concrete during its setting and hardening process. Particular objects include providing for simple, safe and reliable installation of the probe, as well as providing for reliable and user-friendly data collection.

The invention is defined by the appended independent claims, with embodiments being set forth in the appended dependent claims, in the following description and in the attached drawings.

According to a first aspect, there is provided a sensor device for collecting data on a material in which the sensor device is inserted, the sensor device comprising an elongate sensor body, extending along a longitudinal direction, and having a head end and a tip end, at opposite axial ends of the sensor body, at least two first sensor elements, which are spaced apart along the longitudinal direction, both of which being configured for sensing at least one first parameter relating to the material at a respective position along the longitudinal direction, and a locking surface, which faces towards the head end of the sensor body, for interaction with the material.

The material may in particular be concrete. In particular, the sensor device is suitable for collecting data on concrete during its setting and hardening process.

By spacing the sensor elements apart along the longitudinal direction, the first parameter may be sensed at two different depths of the material.

The first parameter is the same at both positions along the longitudinal direction. Hence, it is possible to derive a profile of the parameter along the longitudinal direction.

The first parameter may be humidity or temperature.

The locking surface facilitates insertion of the probe into the material also when the material is not entirely liquid. The locking surface counteracts undesired expulsion of the probe, as may be caused by its buoyancy in the material.

At least part of the sensor body may tapers towards the tip end.

The locking surface may be formed on an exterior thread, which extends substantially helically along at least a portion of the sensor body.

A thread may facilitate insertion of the sensor device also into a material that has begun to set, dry or harden.

The exterior thread may extend along an axial length of the sensor body corresponding at least to an axial length between the sensor elements.

The thread presents an inward flank, facing radially outwardly and towards the tip end and an outward flank, facing radially outwardly and towards the head end, wherein the inward flank and the outward flank present different angles relative to the longitudinal direction.

The inward flank may present a smaller angle than the outward flank.

The thread may be formed by a ridge, which protrudes from an outer surface of the sensor body.

Alternatively, or as a supplement, the thread may be formed by a groove which is recessed into the sensor body.

The head end may present a gripping structure, designed for transferring a torque about an axis which is parallel with the longitudinal direction.

The gripping structure may be adapted for engagement with a torque tool, such as a wrench or a screw driver. To this end, the gripping structure may be formed with a suitable torque transfer interface, such as a square, hexagonal or torx-like shape.

Alternatively, or as a supplement, the locking surface may be formed on a protrusion on the sensor body, such as a ridge or a barb.

Alternatively, or as a supplement, the locking surface may be formed as a recess in the sensor body, such as a groove or dimple.

The head end may comprise a sacrificial material portion, adapted for being removed once the sensor has been inserted into the material.

The sacrificial material portion may be a portion which presents a greater axial wall thickness and/or which presents a protrusion that is suitable for being ground away. Alternatively, the sacrificial material portion may be adapted for being removed, such as by being broken off. To this end, a rupture mark may be provided at a transition portion from the sacrificial material portion to the head end portion.

The sacrificial material portion may comprise at least two material portions, which are visually different from each other and juxtaposed in the longitudinal direction, such that removing one of the material portions exposes the other one of the material portions.

The aspects relating to the sacrificial material portion can be used in sensor bodies not having any external thread. Such sensor bodies may be inserted by a linear movement of the sensor device into the material. A sensor body not having a thread may, but need not, instead have protrusions or depressions on its body, into which wet or unhardened material may flow so as to lock the sensor device into place.

Each of the sensor elements may be operative in a respective sensor space enclosed in the sensor body, and wherein each of the sensor spaces is in communication with an exterior of the sensor body through a semi-permeable membrane.

Each of the sensor spaces may communicate with the exterior through a sensor window, which may present at least two window portions that are separated by a membrane guard.

The semi-permeable membrane may be an expanded PTFE film.

The sensor spaces may be sealed from each other.

The aspects relating to the use of a membrane, a membrane protecting portion and the division of the sensor body into sensor spaces can be used in sensor bodies not having any external thread. Such sensor bodies may be inserted by a linear movement of the sensor device into the material. A sensor body not having a thread may, but need not, instead have protrusions or depressions on its body, into which wet or unhardened material may flow so as to lock the sensor device into place.

The sensor body may have a length along the longitudinal direction, wherein a first one of the sensor elements may be operable at a distance from the tip end, which corresponds to less than about 30% of the length, preferably less than about 20% of the length or less than about 10% of the length.

A second one of the first sensor elements may be operable at a distance from the tip end, which corresponds to about 40-60% of the length, preferably about 45-55% of the length.

A third one of the first sensor elements may be operable at a distance from the tip end, which corresponds to about 70-90% of the length, preferably about 75-85% of the length.

The sensor device may further comprise a controller, a communication device and a power source, wherein the controller is operatively connected to the sensors to receive sensor signals, and to the communication device to communicate data representing said sensor signals.

The communication device may be a wireless data communication device, such as a wifi, Bluetooth, NFC, or the like, type data communication device. Alternatively, the data communication device may be a wired data communication device, using a standardized wired data communication protocol, such as USB, RS232, or the like.

The sensor device may further comprise a data memory, for storing data representing, or derived from, said sensor signals.

Hence, data can be stored in the memory, such that data transfer can be performed at intervals, or upon the sensor device being polled by an external unit.

The first parameter may be selected from a group consisting of humidity, temperature, chloride concentration and vibration.

The sensor device may further comprise at least one second sensor element for sensing a second parameter selected from the group consisting of humidity, temperature, chloride concentration and vibration, said second parameter being different from the first parameter.

In particular, where the first sensor elements are arranged to measure humidity, the second sensor elements may be arranged to measure temperature.

The second sensor element may be positioned at substantially the same position along the longitudinal direction as the first sensor element, preferably one second sensor element at each first sensor element.

The sensor device may comprise at least three first sensor elements, which are distributed along the longitudinal direction.

At least two of the sensor elements may be arranged on a common circuit board, preferably on a same face of the circuit board.

The circuit board may support at least one electronic component in addition to the sensor elements, said electronic component being arranged on an opposite face of the circuit board as compared to at least one of the sensor elements. Preferably, all such electronic components may be arranged on a first face of the circuit board and all sensor elements on a second face of the circuit board.

A surface of the circuit board which is exposed to a sensor space may be covered with a metal layer configured to prevent moisture from penetrating into the circuit board.

According to a second aspect, there is provided a method of collecting data on concrete, comprising providing the concrete in a wet state to a forming site, while the concrete is the wet state, inserting at least one sensor device as claimed in any one of the preceding claims into the concrete, such that the sensor body extends into the concrete and the head end is exposed at a concrete surface, wherein said inserting comprises moving the sensor device about an axis which is parallel with its longitudinal direction, and/or back and forth along the longitudinal direction, so as to cause some of the concrete to interact with the locking surface.

The sensor device may be arranged substantially vertically into the concrete, such that the axially exposed head end surface is substantially horizontal.

The method may further comprise removing at least some material from the head end of the sensor body, such that an axially exposed head end surface of the sensor body is flush with a surrounding concrete surface.

Said removing may comprise removing at least one upper material layer from the head end, so as to expose an underlaying material layer, which is visually different from the upper material layer.

The method may further comprise receiving a series of measurement data provided by each of the sensors, and using said series of measurement data to estimate at least one of a current curing rate and an expected remaining curing time of the concrete.

### BRIEF DESCRIPTION OF THE DRAWINGS

FIGS. 1a-1h are schematic drawings, which disclose an embodiment of a probe 1.

FIGS. 2a-2d schematically illustrate the application of the probe 1, 1′ into concrete.

FIG. 3 schematically illustrates a head end 102 of the probe 1, 1′.

FIGS. 4a-4c schematically illustrate a tip end 101 of the probe 1, 1′.

FIG. 5 schematically illustrates a system comprising the probe 1, 1′.

FIGS. 6a-6b schematically illustrate an alternative design of a sensor space.

### DETAILED DESCRIPTION

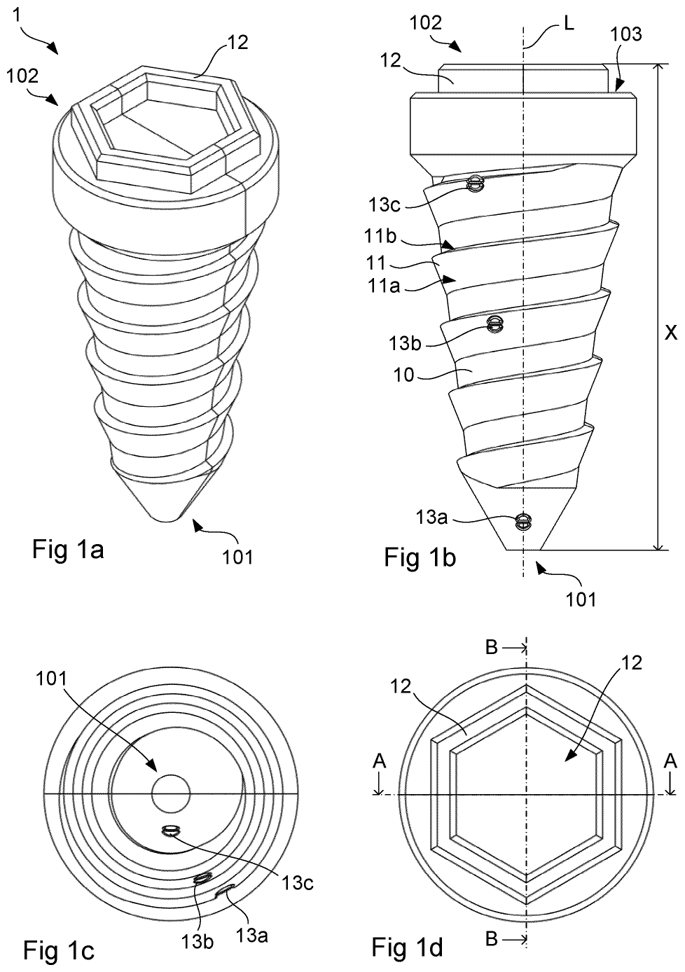

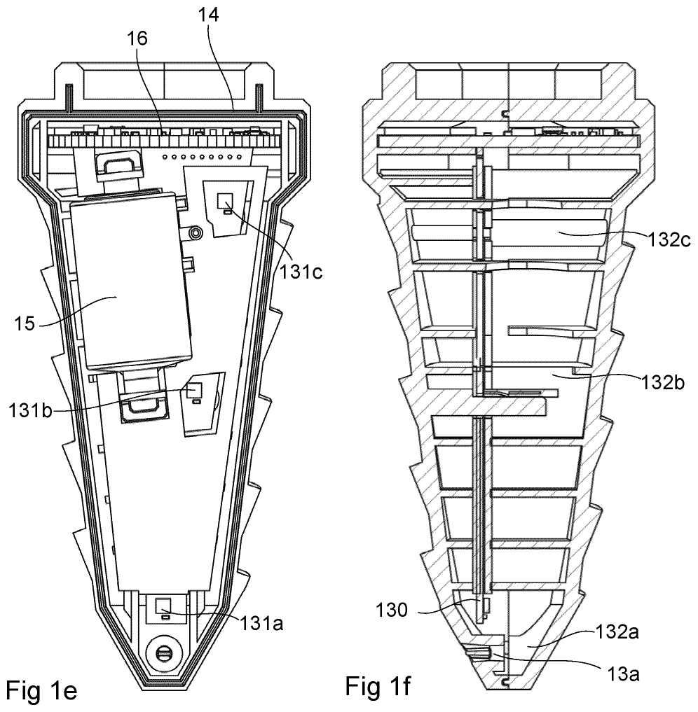

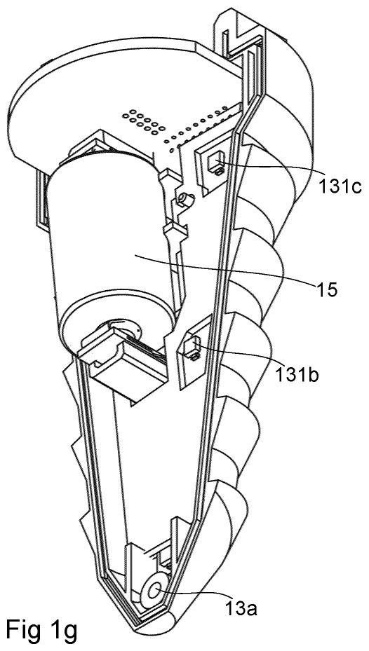

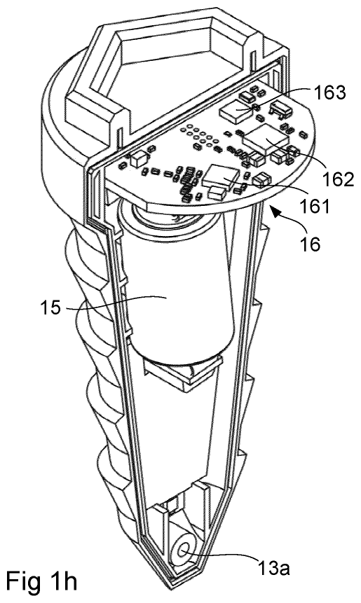

Referring to FIGS. 1a-1h, there is disclosed an embodiment of a probe 1 for collecting data on curing concrete.

The probe 1 comprises a probe body 10, having a tip end 101 and a head end 102, which are arranged at opposite axial ends of the probe body 10 taken along an insertion direction, which may coincide with a longitudinal direction L of the probe body 10. It is understood, that while the probe body 10 is illustrated as an elongate body, having a main extent along the insertion direction/longitudinal direction, the teachings of this document may also be applied to other geometric shapes of the probe body, provided, however, they present an insertion direction.

The probe body 10 may present a generally tapering portion, which tapers in a direction from the head end 102 towards the tip end 101.

The probe 1 may present a gripping structure, for preventing the probe body 10 from leaving a material into which it has been inserted, such as an external thread 11, which extends helically about a thread portion of the probe body 10. The thread 11 may extend along all or most of the tapering portion.

The thread 11 may be formed with an inward flank 11a facing generally radially outwardly and towards the tip end 101, and an outward flank 11b facing generally radially outwardly and towards the head end 102.

In various embodiments, flank angles may be varied.

In the illustrated example, the inward flank 11a presents a smaller angle to the longitudinal direction L, as seen in a plane containing the longitudinal direction L, than the outward flank 11b.

However, the flank angles of the inward and outward flanks 11a, 11b may be equal, or the inward flank angle may be greater than the outward flank angle, if desired.

As alternatives to the thread, the probe body may be provided with one or more protrusions or recesses, which provide at least one locking surface that faces towards the head end 102, for engagement with the material or prevent the probe body from leaving the material.

Along the probe body 10, there are positioned two or more sensor openings 13a, 13b, 13c. In the illustrated example, there are three sensor openings, but further sensor openings may be provided, if desired.

The tip end 101 may be more or less pointed, conical, frusto-conical or rounded, as desired.

The head end 102 may be provided with a gripping structure 12, which is suitable for transferring a torque to the probe body 10. To this end, the gripping structure may be formed as a bolt for engagement by a wrench or the like, or for engagement with a key, which may have an elongate, star-shaped or polygonal interface.

Alternatively, or a as a supplement, the head end 102 may be adapted to receive a force substantially along the insertion direction.

The gripping structure 12 may thus be formed as a protrusion from a head end surface 103. Alternatively, the gripping structure may be formed recessed into the head end surface 103.

In FIG. 1d, there is defined two cross sections A-A and B-B, which both contain the longitudinal direction L. The first cross section A-A coincides with a joint plane, along which two probe body parts are joined to form the probe body. The second cross section B-B is perpendicular to the first cross section.

Hence, the probe body 11 may be formed from two or more probe body parts, which are joined along a plane containing the longitudinal direction L The joint may comprise a seal 14 that may be provided by a sealing strip or by a sealing compound, such that the sensor body 10 is sealed from its exterior, except for at the sensor openings 13a, 13b, 13c.

FIG. 1e illustrates a cross sectional view of the probe body 10 taken along the first cross section A-A.

FIG. 1f illustrates a cross sectional view of the probe body taken along the second cross section B-B.

As illustrated in FIGS. 1b and 1c, the probe may have three sensor openings 13a, 13b, 13c, each sensor opening is covered by a semi-permeable membrane 133a (FIG. 4c), which allows water vapor but not liquid water to pass, such as an expanded PTFE film, which is available under the Gore-Tex® trademark.

Inside each sensor opening 13a, 13b, 13c, there is provided at least one sensor element 131a, 131b, 131c, which is operable in a sensor space 132a 132b, 132c and thus may be spaced from the membrane 133a.

Inside the probe body 10, there may be provided an elongate circuit board 130, such as a printed circuit board, on which all electronics may be arranged.

The circuit board 130 may extend along substantially the entire length of the probe body 10.

A sensor element 131a, 131b, 131c may be configured to measure at least one parameter, such as humidity, temperature, or a chemical parameter, such as chloride concentration. In addition, further sensor elements, such as an accelerometer or a gyro, may be provided in the controller 16 and configured to measure one or more parameters that is common for the entire probe body 10.

The sensor elements 131a, 131b, 131c may be arranged on the circuit board 130 and thus connected to other electronic components, such as semiconductors, resistors, capacitors, or the like, by conductive traces on the circuit board 130.

Two or more of the sensor elements 131a, 131b, 131c may thus be carried by the same circuit board 130.

Each sensor opening may comprise a through hole extending through the wall of the probe body 10 and a guard portion 134a (FIG. 4c), which operates to protect the membrane 133a from being pierced by an object forming part of the concrete, such as a small stone. The guard portion 13a may extend across the sensor opening 13a, 13b, 13c so as to divide the sensor opening into at least two opening portions, which are separated by the guard portion 13a. To this end, the guard portion 13a may be formed as a bar or grille extending across the sensor opening 13a, 13b, 13c. The bar or grille may be formed in one piece with the probe body 10, or it may be formed by a separate part that is attached or integrated with the probe body 10.

Referring to FIGS. 1e, 1g, 1h, the probe body 10 may enclose a power source 15, such as a battery, a controller 16, which may comprise a processor 161, a data memory 162 and a communication device 163 for communication with an external device, preferably wirelessly. The communication device 163 may also be equipped with a geographic tracking device, to enable a position of the probe to be determined. The tracking device may use any known tracking mechanism, including local tracking mechanisms, such as network access points and global tracking mechanisms, such as GPS, Glonass, etc.

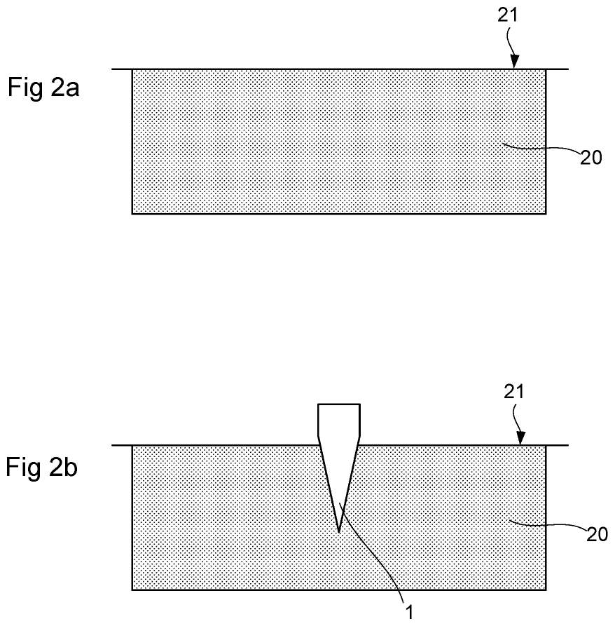

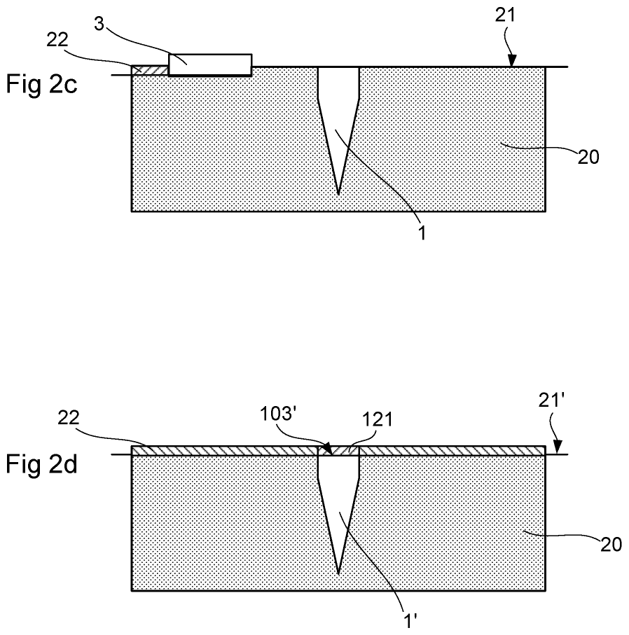

FIGS. 2a-2d schematically illustrate a method of installing the probe 1 in wet concrete.

In FIG. 2a, there is illustrated a body 20 of wet concrete having a surface 21. The body 20 may be a concrete slab or other base for construction. Alternatively, the body 20 may be a wall or any other structure and may comprise reinforcement, additives or the like.

In FIG. 2b, there is illustrated the insertion of the probe 1 into the still wet body 20 of concrete.

The insertion may be achieved by simply pressing the probe 1 into the wet concrete. Alternatively, the probe 1 may be twisted about an axis which is parallel with the longitudinal direction L while inserted into the body 20, such that the threads are used to facilitate the insertion. During this step, the probe may be inserted to an extent such that the head end surface 103 is flush with the surrounding concrete surface 21. Thus, the gripping structure 12 may protrude from the surrounding concrete surface 21.

It is also possible to insert the probe by a linear motion along the insertion direction. Such linear insertion may be supplemented by a twisting motion about the longitudinal direction when the probe has reached its fully inserted position. The linear insertion may also be supplemented by a back-and-forth motion along the longitudinal direction and/or by a vibrating motion about or along the longitudinal direction.

Alternatively, the probe may be inserted such that the axially outermost portion of the gripping structure 12 is flush with the surrounding concrete surface 21.

In FIG. 2c, there is illustrated a grinding step, whereby a grinding tool 3 is used to grind the concrete surface 21 so as to smooth or flatten the concrete surface 21 to form a finished concrete surface 21′. The grinding is carried out such that some material 22 is removed from the concrete surface.

During this grinding step, some material 122 is also removed from the head end portion 102 of the probe body 10, such that a new head end surface 103′ is formed that is flush with the surrounding finished concrete surface 21′, as illustrated in FIG. 2d.

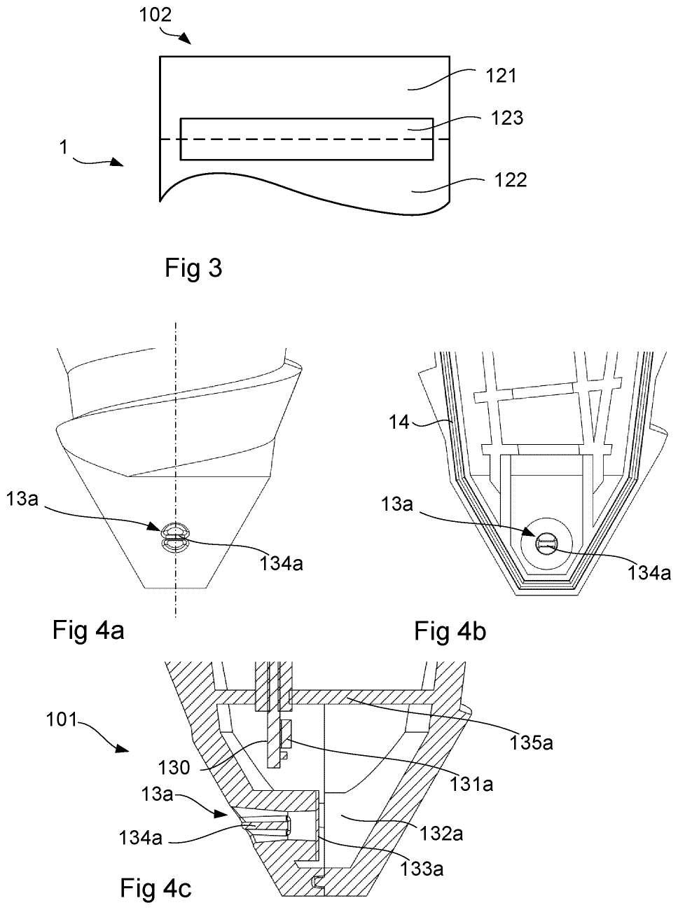

Referring to FIG. 3, there is illustrated how the head end 102 of the probe body may be formed with a sacrificial material portion 121, that is designed to be ground away during the grinding as illustrated in FIG. 2c, so as to leave a remaining part 122 of the head end 102.

The head end may be formed with an indicator material portion 123 that is visually different from the surrounding material 121, 122, such that when the sacrificial material portion 121 is removed, the indicator material portion is exposed to indicate that the probe body 10 has been ground down.

Thicknesses along the longitudinal direction of the sacrificial material portion 121 and of the indicator material portion 123 may be selected so that the indicator material portion 123 serves as a warning not to grind further.

The indicator material portion may also comprise two or more layers, which may also be visually different, so as to indicate, by an upper portion thereof, an allowable degree of grinding, and by a lower portion thereof, an excessive degree of grinding.

As an alternative, or supplement, to using a sacrificial material portion 121, the gripping structure 12 (FIGS. 1a-1h) may be removed, whereby the head end surface 103 may be arranged flush with the concrete surface 21′. To facilitate, a rupture mark may be provided at the transition between the head end surface 103 and the gripping structure, such that the gripping structure may be removed by knocking it sideways relative to the longitudinal direction L, possibly assisted by a tool, such as a hammer, a chisel or a screw driver.

Referring to FIGS. 4a-4c, there is illustrated a tip end 101 of a probe body 10 with the first sensor opening 13a being visible from outside (FIG. 4a), from inside (FIG. 4b) and in cross section (FIG. 4c). The other sensor openings 13a, 13b, 13c and sensor spaces 132a, 132b, 132c may be designed in the same manner as the first sensor opening 13.

With a circuit board 130 to which two or more sensor elements 131a, 131b, 131c are attached, and which extends along the length of the probe body 10, portions of the circuit board at least in the vicinity of each sensor element may be exposed to the respective sensor space 132a, 132b, 132c. Seals may be provided to seal against the circuit board to enclose the respective sensor space 132a, 132b, 132c.

As is illustrated in FIGS. 4a and 4b, the guard portion 134a may be designed as a member extending across the sensor opening 13a, dividing the opening 13a into at least two separated opening portions.

As is illustrated in FIG. 4c, the membrane may be recessed from the outer surface of the probe body. An outermost portion of the guard portion may be substantially flush with the outer surface of the probe body at the sensor opening 13a. An innermost portion of the guard portion 134a may be spaced from the membrane 133.

Inside the sensor space 132a, the sensor element 131a may be spaced from the membrane. In particular, the sensor element 131a (or each sensor element, as the case may be) may be spaced from the membrane in a first direction, which is parallel with the longitudinal direction of the probe body 10. Optionally, the sensor element 131a (or each sensor element, as the case may be) may be spaced from the membrane in a direction perpendicular to the longitudinal direction.

As can be seen in FIG. 4c, the sensor space 132a may be sealed from other parts of the probe body 10 by at least one inner wall 135a.

Hence, the sensor body may enclose two or more sensor spaces, which are sealed from each other, each of which enclosing one or more sensor elements 131a, 131b, 131c. The sensor element or elements arranged in one sensor space may communicate with the outside of the probe body 10 through the same sensor opening 13a, 13b, 13c.

A probe 1, 1′, installed as disclosed with reference to FIGS. 2a-2d, may be operated as follows.

The probe 1, 1′ may be delivered from the supplier in an active state. Alternatively, the probe 1, 1′ may be activated by the operator in connection with its installation. Activation may take place in many different ways, including pressing a button; performing a gesture or otherwise subjecting the probe 1, 1′ to a certain movement or impact; or by wireless communication with an external device, which may be a handheld device, such as a cellular phone, a smartphone or a tablet computer, or a network device, such as a network access point. Activation may also be performed using a dedicated device, which may communicate wirelessly with the probe 1, 1′.

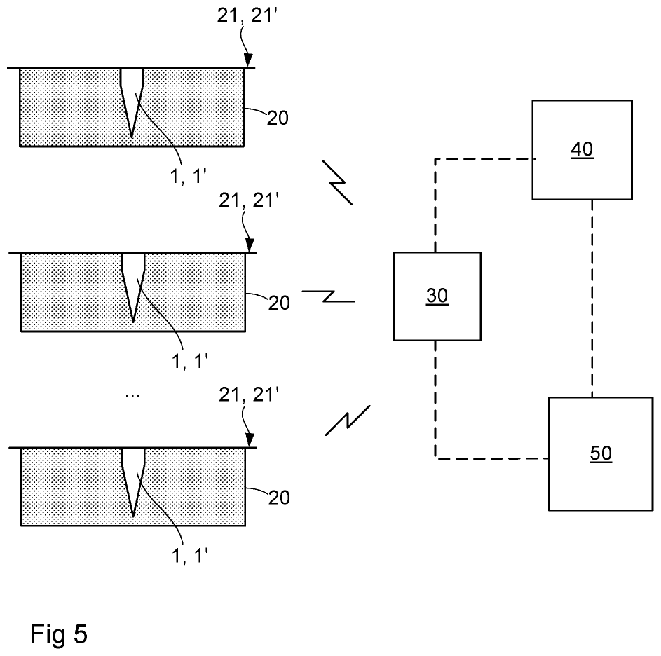

Referring to FIG. 5, once the probe is activated, it may be caused to run a predetermined measurement and reporting scheme, whereby measurements are made at predetermined time intervals, as desired.

Measurement data may be temporarily stored in the memory 162 of the controller 16, such that it can be accessed by, or otherwise communicated to, an external unit 30 by means of the communication device 163. The external unit 30 may be a network access point or any other communication device suitable for receiving data from the probes 1, 1′. Such data transfer may be initiated by the probe 1, 1′ or by the external unit 30.

Measurement data may thus be communicated to the external unit 30 at predetermined time intervals, and may be synchronized with the times at which measurements are made.

The measurement data may be received by the external unit 30 and forwarded to a storage device 40, from which it may be accessed by a monitoring device 50 and/or by a user.

The monitoring device 50 may be configured to run various monitoring processes, e.g. to determine curing rate and to estimate curing time and/or a point in time when the concrete is sufficiently cured for further processing thereof, or construction thereon to be allowable.

The determination of curing rate and/or curing time may also be based on further parameters, such as air temperature in the surrounding environment, air humidity and/or light intensity from e.g. sun light. Such factors may be determined in a per se known manner by one or more additional sensors positioned in the vicinity of the probes 1, 1′.

The monitoring device may also provide an alarm in the event measurements indicate that the curing process deviates from expectations, e.g. due to unexpected variations in measurement parameters.

An alarm may also be provided in the event an error indication is received from a probe, and/or in the event a probe fails to communicate as expected.

An indication may also be provided in response to the occurrence of a predetermined event, such as the completion of the curing process and/or the achieving of a predetermined state, such as strength, of the concrete.

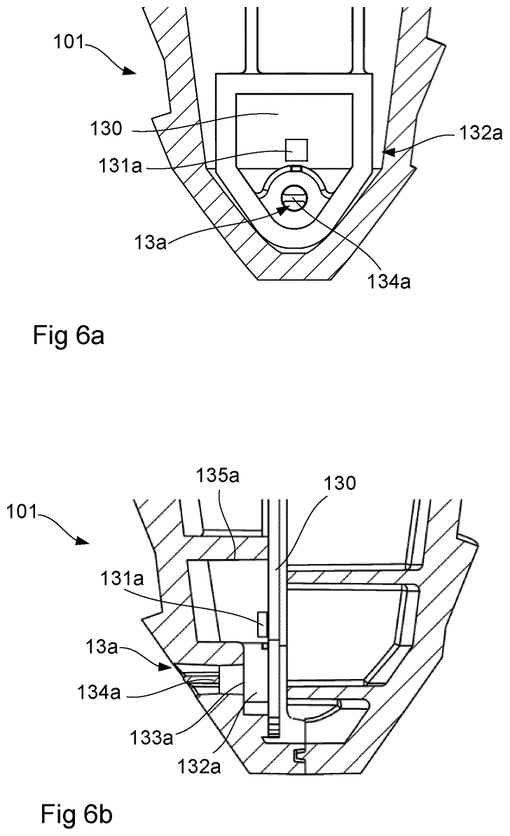

FIGS. 6a-6b schematically illustrate an alternative design of a sensor space 132a, in which the sensor element 131a has been arranged on a back side of the circuit board 130, instead of on the front side, where other electronic components are arranged.

In either embodiment, surfaces of the circuit board 130 which are exposed to a sensor space 132a, 132b, 132c may be coated with a metal layer, such as, but not limited to, copper, in order to prevent water from contacting the circuit board 130.

1. A sensor device for collecting data on concrete during its setting and hardening process, in which the sensor device is inserted, the sensor device comprising:
an elongate sensor body, extending along a longitudinal direction, and having a head end and a tip end, at opposite axial ends of the sensor body;
at least two first sensor elements, which are spaced apart along the longitudinal direction, both of which being configured for sensing at least one first parameter relating to the material at a respective position along the longitudinal direction, and
a locking surface, which faces towards the head end of the sensor body, for engagement with the concrete.

2. The sensor device as claimed in claim 1, wherein at least part of the sensor body tapers towards the tip end.

3. The sensor device as claimed in claim 1, wherein the locking surface is formed on an exterior thread, which extends substantially helically along at least a portion of the sensor body.

4. The sensor device as claimed in claim 3, wherein the exterior thread extends along an axial length of the sensor body corresponding at least to an axial length between the sensor elements.

5. The sensor device as claimed in claim 3, wherein the thread presents an inward flank, facing radially outwardly and towards the tip end and an outward flank, facing radially outwardly and towards the head end, wherein the inward flank and the outward flank present different angles relative to the longitudinal direction.

6. The sensor device as claimed in claim 5, wherein the inward flank presents smaller angle than outward flank.

7. (canceled)

8. (canceled)

9. The sensor device as claimed in claim 1, wherein the head end presents a gripping structure, designed for transferring a torque about an axis which is parallel with the longitudinal direction.

10. (canceled)

11. (canceled)

12. The sensor device as claimed in claim 1, wherein the head end comprises a sacrificial material portion, adapted for being removed once the sensor device has been inserted into the material.

13. The sensor device as claimed in claim 12, wherein the sacrificial material portion comprises at least two material portions, which are visually different from each other and juxtaposed in the longitudinal direction, such that removing one of the material portions exposes the other one of the material portions.

14. The sensor device as claimed in claim 1, wherein each of the sensor elements is operative in a respective sensor space enclosed in the sensor body, and wherein each of the sensor spaces is in communication with an exterior of the sensor body through a semi-permeable membrane.

15. The sensor device as claimed in claim 14, wherein each of the sensor spaces communicates with the exterior through a sensor window, which presents at least two window portions that are separated by a membrane guard.

16. (canceled)

17. The sensor device as claimed in claim 14, wherein the sensor spaces are sealed from each other.

18. (canceled)

19. (canceled)

20. (canceled)

21. The sensor device as claimed in claim 1, further comprising a controller, a communication device and a power source, wherein the controller is operatively connected to the sensor elements to receive sensor signals, and to the communication device to communicate data representing said sensor signals.

22. (canceled)

23. The sensor device as claimed in claim 1, wherein the first parameter is selected from a group consisting of humidity, temperature, chloride concentration and vibration.

24. (canceled)

25. (canceled)

26. The sensor device as claimed in claim 1, wherein the sensor device comprises at least three first sensor elements, which are distributed along the longitudinal direction.

27. (canceled)

28. (canceled)

29. (canceled)

30. A method of collecting data on concrete, comprising:
providing the concrete in a wet state to a forming site,
while the concrete is in the wet state, inserting at least one sensor device as claimed in any one of the preceding claims into the concrete, such that the sensor body extends into the concrete and the head end is exposed at a concrete surface,
wherein said inserting comprises moving the sensor device about an axis which is parallel with its longitudinal direction, and/or back and forth along the longitudinal direction, so as to cause some of the concrete to interact with the locking surface.

31. The method as claimed in claim 30, wherein the sensor device is arranged substantially vertically into the concrete, such that an axially exposed head end surface is substantially horizontal.

32. The method as claimed in claim 30, further comprising removing at least some material from the head end of the sensor body, such that an axially exposed head end surface of the sensor body is flush with a surrounding concrete surface.

33. The method as claimed in claim 32, wherein said removing comprises removing at least one upper material layer from the head end, so as to expose an underlaying material layer, which is visually different from the upper material layer.

34. The method as claimed in claim 30, further comprising receiving a series of measurement data provided by each of the sensors, and using said series of measurement data to estimate at least one of a current curing rate and an expected remaining curing time of the concrete.
